(19) United States
(12) Patent Application Publication
(10) Pub. No.: US 2015/0038963 A1
(43) Pub. Date: Feb. 5, 2015

(54) ABLATION CATHETER

(71) Applicant: CATHRX LTD, Homebush Bay, New South Wales (AU)

(72) Inventors: James Panos, Earlwood (AU); Tyler McKinley Mortimer, Bondi Beach (AU); Roman Greifeneder, Bexley (AU)

(21) Appl. No.: 14/388,060

(22) PCT Filed: Mar. 27, 2013
(86) PCT No.: PCT/AU2013/000319

§ 371 Date: Sep. 25, 2014

Related U.S. Application Data
(60) Provisional application No. 61/616,016, filed on Mar. 27, 2012.

(51) Int. Cl. A61B 18/14; A61M 3/02

(52) U.S. Cl. CPC A61B 18/1492; A61M 3/0279; A61B 2018/1472; A61B 2018/00166
USPC 606/41

(57) ABSTRACT

An ablation catheter including a handle, a tubular irrigation member defining a fluid lumen and having a plurality of perforations proximal the distal end of the tubular irrigation member. At least one ablation electrode is arranged at the distal end of an elongate tubular sheath inserted into the lumen of the tubular irrigation member, the elongate tubular sheath being axially displaceable within the lumen of the tubular irrigation member. The ablation catheter may also include two ablation electrodes each attached to an elongate tubuar sheath which are telescopically arranged within the tubular irrigation member. The ablation electrodes may be displaceable simultaneously together or individually.

### TECHNICAL FIELD

This disclosure relates, generally, to a catheter and, more particularly, to an ablation catheter.

### BACKGROUND

Any discussion of the prior art throughout the specification should in no way be considered as an admission that such prior art is widely known or forms part of common general knowledge in the field.

In the conduction of Maze type procedures, an ablation catheter is used to ablate heart tissue to attempt to clear heart arrhythmias. Generally a dot ablation is made and this is repeated by re-positioning a tip and the ablation electrode of an ablation catheter. This is an extremely time consuming process. In addition, dot ablation may leave gaps in the lesions which may again require re-positioning and repeating the procedure. If a clinician could form longer lesions, fewer manipulations would be required. This would reduce the time to conduct the procedure which would be beneficial for all concerned. Longer electrodes have been considered for radiofrequency ablation but coagulum tends to form on the electrodes. In addition, the energy field from long electrodes is not always uniform and this may cause discontinuities in the lesion. Furthermore, the temperature of the ablation electrodes as well as the tissue being treated needs to be carefully maintained to ensure that it does not result in excessive ablation of the tissue.

### SUMMARY

It is an object of the present invention to overcome or ameliorate at least one of the disadvantages of the prior art, or to provide a useful alternative.

In an aspect, an ablation catheter is provided which includes a tubular irrigation member of a non-conductive material having a proximal end and a distal end, the tubular member defining a fluid lumen and having one or more perforations at or proximal the distal end of the tubular member, an elongate tubular sheath having a lumen extending from a proximal end of the tubular sheath to a distal end of the tubular sheath, and one or more ablation electrodes arranged at, or adjacent, the distal end, the elongate tubular member being removably received into the fluid lumen of the irrigation member and the proximal end of the elongate tubular sheath being connected to a control handle, wherein the elongate tubular sheath is axially displaceable within the fluid lumen of the irrigation member.

In an embodiment, the catheter includes a second elongate tubular sheath having one or more ablation electrodes arranged at, or adjacent, its distal end, the second elongate tubular sheath being axially displaceable within the lumen of the irrigation member. The one or more ablation electrodes of the first or the second tubular sheath overlap with the one or more perforations of the irrigation member.

In an embodiment, the displacement of the first or the second elongate tubular sheath is motorized.

In an embodiment, the second elongate tubular sheath is telescopically arranged around the first elongate element.

In an embodiment, the tubular irrigation member includes an inlet for receiving pressurized fluid into the fluid lumen of the irrigation member to be energized by the one or more ablation electrodes of the first or the second elongate tubular sheath before being expelled through the one or more perforations on the irrigation member. The proximal end of the tubular irrigation member also includes a seal to be in a sealing connection with the first or the second tubular sheath.

In an embodiment, the proximal end of the tubular irrigation member includes a locking mechanism for releasably connecting the irrigation member to the control device.

In an embodiment, the first and the second elongate tubular sheath are axially displaceable in relation to one another. When the first and the second elongate tubular sheath include two or more electrodes these electrodes can be arranged at a predetermined distance from one another.

In an embodiment, the distal end of the tubular irrigation member is heat set into a predetermined shape. Preferably, the predetermined shape is a loop shape. In addition, a steering element may be inserted into the lumen of the first or the second elongate tubular sheath.

In an embodiment, the perforations on the irrigation member are arranged in a predetermined pattern.

In an embodiment, conductors for the electrodes are contained within a wall of the tubular member. Preferably, the conductors are wound helically between an inner and an outer layer of non-conductive material.

In an embodiment, there is provided an ablation catheter which includes a tubular irrigation member of a non-conductive material having a proximal end and a distal end, the tubular member defining a fluid lumen and having one or more perforations at or proximal the distal end of the tubular member, an elongate tubular sheath having a lumen extending from a proximal end of the tubular sheath to a distal end of the tubular sheath, the distal end of the elongate tubular sheath formed into a loop shape, the elongate tubular sheath having a plurality of ablation electrodes arranged at, or adjacent, the distal end, the elongate tubular member being removably received into the fluid lumen of the irrigation member and imposing the loop shape onto the irrigation member, and a control handle, the proximal end of the elongate tubular sheath further being connected to a control handle.

The proximal end of the tubular irrigation member includes an irrigation connector for connecting the irrigation member to the elongate tubular sheath. This irrigation connector includes a sealing element and an inlet for receiving pressurized fluid into the fluid lumen of the irrigation member to be energized by the one or more ablation electrodes of the first or the second elongate tubular sheath.

### BRIEF DESCRIPTION OF DRAWINGS

Preferred embodiments of the invention will now be described, by way of example only, with reference to the accompanying drawings in which:

FIG. 1 shows a perspective view of a distal part of a catheter sheath of an ablation catheter where only one ablation electrode is arranged in a lumen of the sheath;

FIG. 2 shows a perspective view of a distal part of a catheter sheath of an ablation catheter where two ablation electrodes are arranged in a lumen of the sheath;

FIG. 3 shows a schematic side view of a looped ablation catheter;

FIG. 4 shows a cross sectional view of the catheter sheath with two ablation electrodes;

FIG. 5a-5c show a loop catheter, a tubular irrigation member and the catheter when the tubular irrigation member is connected to the catheter;

FIG. 6a-6c show a catheter with a deflective tip, a tubular irrigation member, and the catheter when the tubular irrigation member is inserted onto the catheter; and

FIGS. 7a and 7b show a catheter being inserted into an irrigation inserter having a handle for deflecting the distal tip of the inserter.

### DESCRIPTION OF EMBODIMENTS

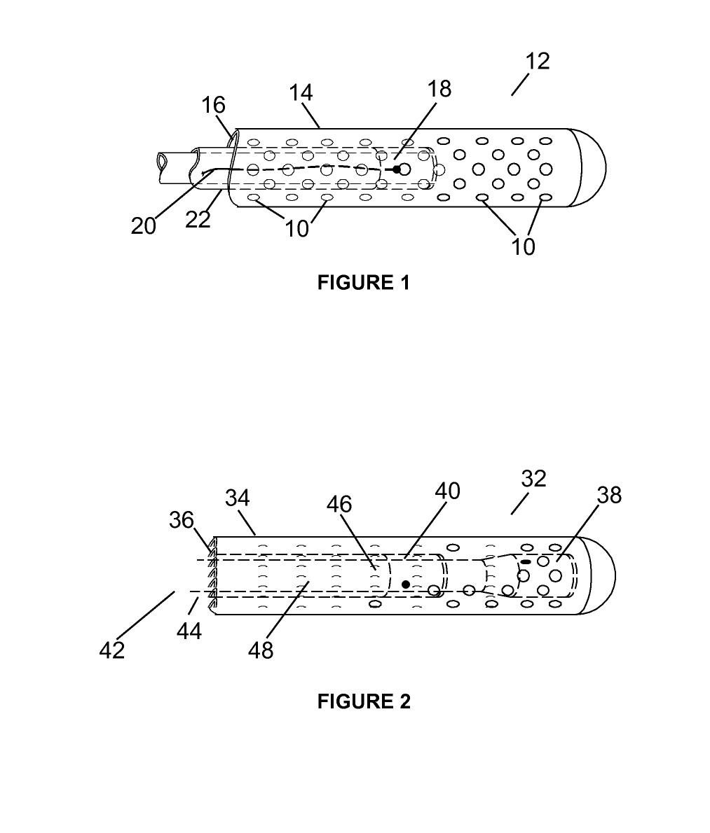

FIG. 1 of the drawings depicts the distal end 12 of an ablation catheter. The outer irrigation member 14 is a sheath that defines a fluid lumen 16 that extends from the proximal end of the irrigation member to the distal end of the irrigation member. The irrigation member 14 is a tubular member made of a suitable polymeric material such as polyethylene or polyether block amide (PEBAX®). Other suitable, bio-compatible polymeric materials could also be used.

Ablation of tissue is achieved by RF (radiofrequency) energy transfer through electrically charged saline fluid escaping the distal end 12 of the irrigation member 14 through multiple perforations 10. These perforations 10 may populate the entire wall of the irrigation member 14 or they may be arranged at a pre-determined formation on the irrigation sheath so as to direct the fluid path to a desired location or direction. The conductive fluid is supplied to the lumen of the catheter sheath by an irrigation pump via a suitable connector element such as a Luer connector on the catheter control handle or proximal the proximal end of the irrigation member 14. The pressurized fluid is expelled towards the tissue through the multiple perforations 10 in the distal end 12 of the irrigation member 14.

The fluid is energized by supplying the electrode 18 with RF (radiofrequency) energy via the conductor wire 20. The electrode is attached to the distal end of an elongate tubular sheath 22. The conductor wire 20 is electrically connected to the electrode and connects the electrode to an energy source via the catheter control handle (not shown in FIG. 1). When the electrode 18 is supplied by RF energy via the conductor wire 20, the electrode energizes the fluid adjacent the electrode and the energized and pressurized fluid is then expelled to the tissue through the perforations 10 on the irrigation member 14 in the proximity of the electrode. Non-energized fluid is still expelled through the perforations elsewhere in the catheter sheath and this fluid cools the electrodes as well as the ablated tissue.

The elongate tubular sheath 22 and the electrode 18 are axially slidable within the irrigation member 14. This way, the clinician can slide the electrode to make a continuous linear lesion to cause a “drag burn”. The energized fluid squirts out of the perforations 10 on the irrigation member 14 and ablates the tissue along the path of the electrode.

Whilst not depicted in the accompanying drawings, the catheter control handle includes appropriate controls for moving and sliding the electrode 18 and elongate tubular sheath 22 within the irrigation member. The catheter handle may also include control knobs for controlling the deflection or the size of deflection or for controlling the size of the loop in case of a looped catheter. Furthermore, the handle may include appropriate elements for motorized control for moving the electrodes. Although not shown in the accompanying figures, the irrigation member 14 and the control handle include appropriate connector elements to removably attach and detach the irrigation member from the control handle.

FIG. 2 shows the distal end 32 of an irrigation member 34 where two electrodes 38 and 40 are used to energize the fluid within the lumen 36 of the irrigation member 34. Each electrode 38 and 40 is attached to the distal end of a tubular sheath 42 and 44. The elongate tubular sheath 42 slides telescopically within the elongate tubular sheath 44. Each tubular sheath 42 and 44 and hence each electrode 38 and 40 can slide within the irrigation member either individually or simultaneously. One of the electrodes may be anchored to stay in place while the other electrode can slide. In this way, it is possible to change the spacing of the electrodes to a desired distance to cause ablation of the tissue for a desired lesion depth or a desired length. The catheter handle has appropriate controls for controlling the movement of the electrodes.

Two electrodes are typically used if a bipolar type of operation is effected where two, generally adjacent, electrodes are energised simultaneously to cause RF energy flow between the adjacent electrodes. Using two electrodes with spacing allows for phase controlled ablation which provides control of depth of linear lesion in comparison to unipolar ablation with one electrode. Each electrode can be supplied with RF energy via the conductor wires 46 and 48.

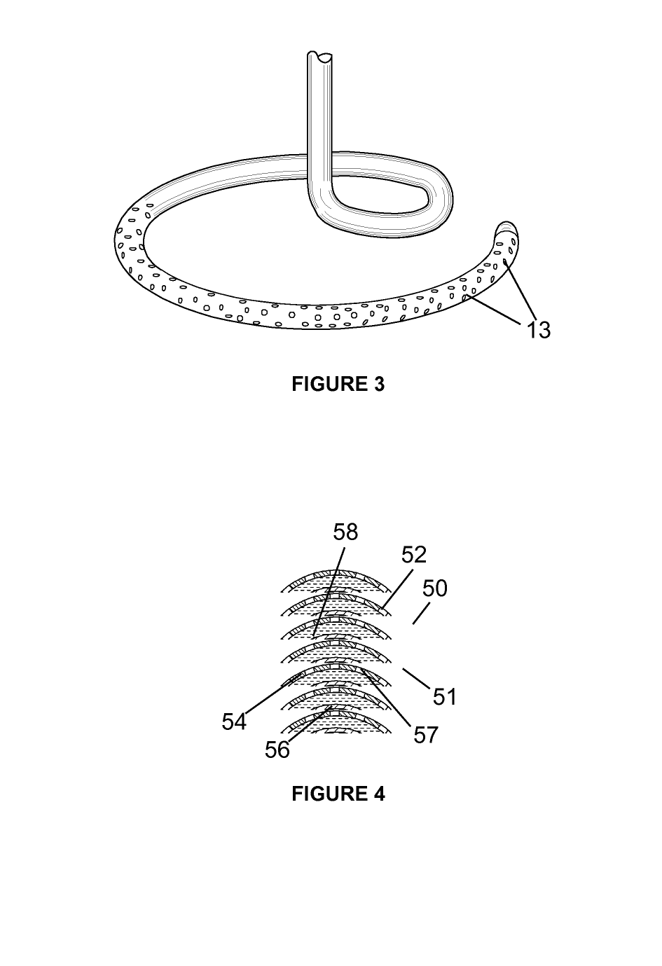

The distal end of the ablation catheter may be of straight configuration as seen in FIGS. 1 and 2. It is also possible that a shape-memory wire is inserted into the lumen of the tubular sheath 22, 42 or 44 that will impart a desired shape to the distal end of the irrigation member. The distal end of the irrigation member may also be heat set to a loop shape as shown in FIG. 3. In addition, the loop size of the distal tip may be varied by increasing or decreasing the size of the loop. In the embodiment depicted in FIG. 3, the perforations 13 are located preferably on the outer surface of the irrigation member so that the energized fluid is better directed towards tissue. Any pattern of the perforations can be used but in a loop embodiment, for example, covering ⅓ of the surface of the sheath is used, preferably the outer surface of the loop. Using directional RF energy by different patterns of the perforations means that lower power levels will be required to create an effective lesion as less energy is lost to the blood pool.

FIG. 4 shows the cross section of the irrigation member 50 of the electrode sheath of an ablation catheter having two telescopic elongate tubular sheaths within the irrigation member. The irrigation catheter may include a steering wire/wires or a stylet 57 that can be used for steering of the catheter to the desired location. The stylet may also allow the user to change the size of the loop at the distal end of the catheter. The lumen 51 is a fluid lumen where the pressurized saline fluid is supplied to be energized by the electrodes attached to the distal ends of the tubular sheaths 52 and 54. Conductor wires 56 and 58 enable energizing of the electrodes. The fluid in the lumen 51 cools down the electrodes. The lumen 51 communicates with a Luer connector arranged near or at a proximal end of the irrigation member or at the control handle for connection to a supply of irrigation fluid (not shown).The irrigation member 50 as well as the elongate tubular sheaths 52 and 54 can be made of light-transparent material.

The movement of the one or two electrodes within the electrode sheath can be motorized so that they slide at a predetermined speed for a predetermined period of time causing a lesion of predetermined length. The control handle includes appropriate controls so that the user may select a desired speed or length. The movement of the electrodes can be set pitch where the distance between the electrodes is fixed or it can be set variable pitch where the distance between the electrodes varies.

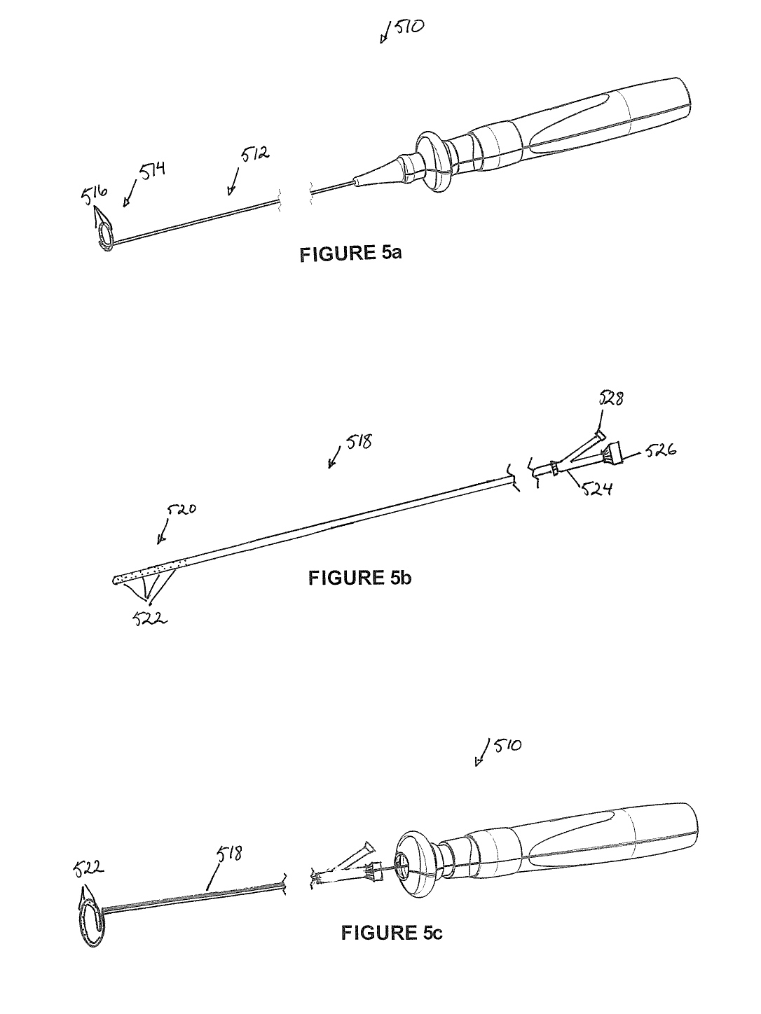

FIG. 5a depicts a catheter 510 having a loop at the distal end of the elongate tubular sheath 512 of the catheter. However, the catheter may have any desired shape at the distal end 514. The distal end 514 may be rectilinear deflective tip or a non-rectilinear configuration such as the loop shown in FIG. 5a or a spiral formation. The catheter has one or more ablating electrodes at the distal end of the sheath 512 around the loop. FIG. 5b shows a tubular irrigation member 518. The irrigation member 518 has a distal end 520 that has multiple perforations 522 extending through the wall of the irrigation member to let fluid pass from inside of the irrigation member out towards the tissue. The perforations 522 may be located evenly or unevenly around the circumference of the distal tip 520 of the irrigation member 518. They can also form a pattern so as to direct the fluid towards the tissue in a desired way. FIG. 5c depicts the catheter 510 when the catheter is inserted into the lumen of the tubular irrigation member 518. The shape of the catheter imposes the same shape on the irrigation member 518. The lumen of the irrigation member 518 is capable of fitting the catheter tubular sheath 512 with enough space in the lumen and around the tubular sheath 512 to allow fluid to flow in the lumen of the irrigation member 518.

The tubular irrigation member 518 also includes an irrigation connector 524. The irrigation connector 524 includes a seal 526 and a lateral fluid inlet 528. When the irrigation member 518 is slid onto the tubular sheath 512 of the catheter fluid can be pushed through the inlet 528 into the lumen of the irrigation member 518 and towards the perforations 522 at the distal and of the irrigation member. The fluid is energized by the ablation electrodes 516 of the catheter as the fluid reaches the distal end of the irrigation member. The energized fluid then expels out through the perforations towards the tissue to be ablated. The perforations are preferably on the outer surface of the loop shaped distal tip 514 such that the pressurized fluid expels out of the perforations radially from the central axis of the tubular sheath 12. The seal 526 is in a sealing engagement with the tubular sheath 512 of the catheter. The seal 526 may an o-ring or a membrane that allows the tubular sheath 612 of the catheter to pass through it but will prevent any backflow of fluid out of the lumen of the irrigation member 618.

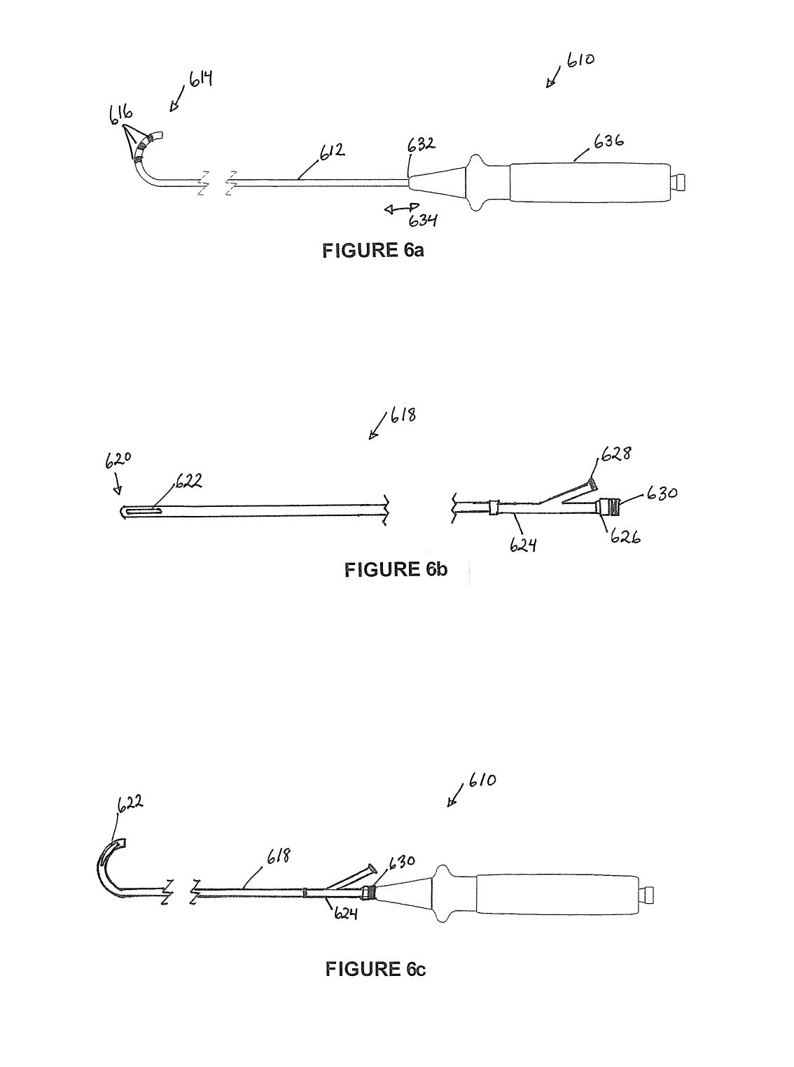

FIGS. 6a-6c show a catheter 610 with a deflective tip 614. The deflective tip 614 has one or more electrodes attached proximal to the distal end 614 of the catheter sheath. The elongate tubular sheath 612 of the catheter and thus the ablation electrodes 616 are movable in a longitudinal direction in the direction of the arrows 634 in FIG. 6a. The connection 632 allows for the elongate tubular sheath of the catheter to move axially in relation to the catheter handle 636. Although not depicted in FIG. 6a or 6c the handle includes appropriate controls for controlling the movement of the tubular sheath in relation to the handle. This movement may also be motorized.

FIG. 6b depicts a tubular irrigation member 618. The irrigation member 618 has one or more openings 622 proximal the distal end 620 of the irrigation member. In the embodiment of FIG. 6b, the irrigation member has one elongated opening extending through the wall of the tubular sheath 618. The opening 622 can also have any other desired shape. Similarly to the irrigation member described in FIG. 5b, the irrigation member has an irrigation connector 624 with a seal 626 and a lateral fluid inlet 628. The irrigation connector further includes a locking mechanism 630 to releasably and detachably connect the irrigation member 618 to the catheter handle 636.

FIG. 6c shows the catheter 610 when it has been inserted into the lumen of the tubular irrigation member 618. As the fluid reaches the tip of the irrigation member the one or more ablation electrodes 616 energize the fluid by RF energy (the energy source not depicted in the accompanying figures) and the fluid is expelled through the opening 622 toward the tissue to be treated. When the irrigation member 618 is connected to the catheter handle 636 the tubular sheath of the catheter can be pulled in the direction of the arrows 634 within the irrigation member 618. The electrodes move inside the irrigation member along the opening 622 and the charged fluid expelling from the opening 622 creates an even lesion minimising or eliminating charring.

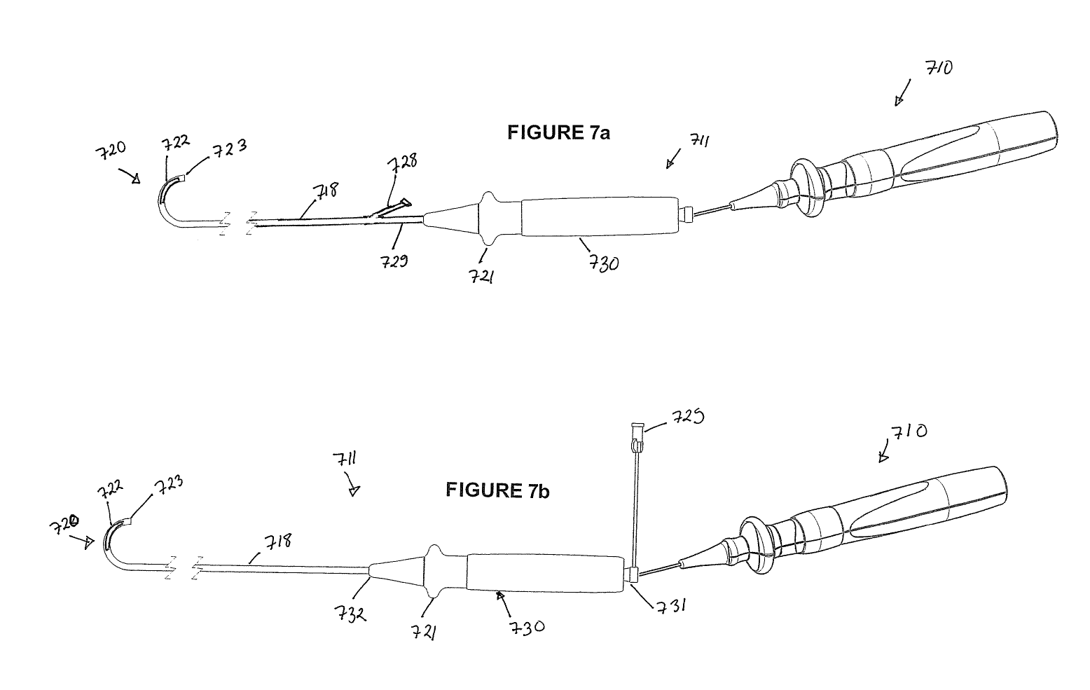

FIGS. 7a and 7b show a catheter 710 being inserted into a catheter inserter 711. A catheter inserter 711 may be used to guide the catheter 710 into the correct physiological treatment area. The inserter 711 includes a deflective distal end 720 and a control 721 for controlling the deflection on the handle 730. The distal tip 723 of the catheter inserter 711 is closed but has an opening 722 similar to the one described in relation to FIG. 6b.

In FIG. 7a, the inserter includes a fluid inlet 728 near the proximal end 729 of the inserter sheath 718. It also includes a seal such as an o-ring or a membrane (not shown in FIG. 7a) to prevent backflow of fluid into the inserter handle 730. In FIG. 7b, the fluid inlet is an appendage 729 at the proximal end 731 of the handle 730. The appendage 729 continues as a passageway through the handle (not shown in FIG. 7b) and allows the fluid to fill the lumen of the inserter sheath 718 after the distal end 732 of the handle when the catheter is in use. The electrode(s) of the catheter 710 energize the pressurized fluid in the lumen of the inserter 711. As the energized fluid expels through the opening 722 towards the tissue it ablates it by creating an even lesion. The lesion length will depend of the size of the opening 722 and the number of electrodes on the electrode sheath of the catheter 710. It is also possible for the user to pull the catheter 710 so that the electrodes slide within the inserter and along the opening 722. In this way, the user is able to create a longer and more even lesion with only one or two electrodes on the catheter sheath.

The tubular sheaths 510, 610 and 710 of the catheter are made of a cable having conductors embedded in non-conductive material leaving a hollow lumen for the electrode sheath. Preferably, the cable consists of a plurality of conductors coiled in a helical manner around an outer surface of an inner non-conductive tubular member. The cable further includes an outer layer of non-conductive material. This allows for a steering element such as a stylet to be inserted into the lumen of the catheter sheath. Electrodes are formed on the surface of the tubular electrode sheath by exposing one of the conductors of the cable and making an electrical connection between the conductor and a surface electrode attached on the outer surface of the catheter sheath. Because the electrode sheath is made of helically wound cable the electrodes can be formed in any desired pattern. In addition, it allows for a large number of electrodes to be formed on the electrode sheath as there will be no conductors running through the lumen of the catheter sheath. For any of the embodiments depicted in the accompanying figures, although the figures describe ablation electrodes, it is naturally possible for some of these electrodes to be sensing electrodes, as necessary.

The electrodes can be arranged to have individual power sources to allow for unipolar construction. In this arrangement, the return electrode would be on the back of the patient. If a bipolar arrangement is desired, the electrodes can be arranged sequentially so that selected electrode/s act as a source electrode and every other electrode acts as a return electrode. In addition, the unipolar and bipolar construction can be combined and a phase difference can be introduced for each electrode. In this configuration, the return electrode for the unipolar energy component would be on the back of the patient.

Using irrigation as a conduit for RF energy minimises edge effects because it virtually increases the size of the electric field source. Edge effects concentrate the RF energy at the edges of the electrodes and cause energy gradients that make the lesion uneven. As there is a constant exchange of irrigation there is constant cooling at the tissue surface whilst continuously delivering energy through the tissue.

In addition, the pressurised fluid within the space between the non-conductive irrigation sheath 518, 618 and 718 and the catheter sheath 512 and 612 with the electrodes 516 and 616 cools the electrodes simultaneously so as to avoid or eliminate charring of tissue. It is an advantage of the described embodiment that an ablation catheter is provided which contains one or more electrodes that can slide within the catheter sheath whilst energizing the fluid in the lumen of the sheath so that tissue surrounding the sheath is ablated by energized fluid through small perforations on the catheter sheath. This allows for creating longer and more even lesions when ablating the tissue.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinarily skill in the art from this disclosure, in one or more embodiments.

As used herein, unless otherwise specified the use of ordinal adjectives “first”, “second”, “third”, etc., to describe a common object, merely indicate that different instances of like objects are referred to, and are not intended to imply that the objects so described must be in a given sequence, either temporally, spatially, in ranking, or in any other manner.

In the claims below and the description herein, any one of the terms comprising, comprised of or which comprises is an open term that means including at lest the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at lest the elements/features that follow the term, but not excluding others. Thus, including is synonymous with and means comprising.

It should be appreciated that in the above description of exemplary embodiments of the invention, various features of the invention are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less that all features of a single foregoing disclosed embodiment. Thus the claims following the Detailed Description are hereby expressly incorporate into this Detailed Description, with each claim standing on its own as a separate embodiment of this invention.

Furthermore, while some embodiments described herein include some but not other features included in other embodiments, combination of features of different embodiments, as would be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected”, along with their derivatives, may be used. It should be understood that these terms are not intended as synonyms for each other. Thus the scope of the expression a device A coupled to a device B should not be limited to devices or systems wherein an output of device is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the invention.

It will be appreciated by persons skilled in the art that numerous variations and/or modifications may be made to the disclosure as shown in the specific embodiments without departing from the scope of the disclosure as broadly described. The present embodiments are, therefore, to be considered in all respects as illustrative and not restrictive.

1. An ablation catheter which includes:
a tubular irrigation member of a non-conductive material having a proximal end and a distal end, the tubular member defining a fluid lumen and having one or more perforations at or proximal the distal end of the tubular member;
an elongate tubular sheath having a lumen extending from a proximal end of the tubular sheath to a distal end of the tubular sheath, and one or more ablation electrodes arranged at, or adjacent, the distal end, the elongate tubular member being removably received into the fluid lumen of the irrigation member and the proximal end of the elongate tubular sheath connected to a control handle, wherein the elongate tubular sheath is axially displaceable within the fluid lumen of the irrigation member.

2. The catheter according to claim 1 in which the tubular irrigation member includes an inlet for receiving pressurized fluid into the fluid lumen of the irrigation member to be energized by the one or more ablation electrodes before being expelled through the one or more perforations of the irrigation member.

3. The catheter according to claim 1 which includes a second elongate tubular sheath having one or more ablation electrodes arranged at, or adjacent, its distal end, the second elongate tubular sheath being axially displaceable within the lumen of the irrigation member.

4. The catheter according to claim 3 where the one or more ablation electrodes of the first or the second tubular sheath overlap at least partially with the one or more perforations of the irrigation member.

5. The catheter according to claim 3 in which the second elongate tubular sheath is telescopically arranged around the first elongate element.

6. The catheter according to claim 3 in which the proximal end of the tubular irrigation member includes a seal to be in a sealing connection with the first or the second tubular sheath.

7. The catheter according to claim 1 in which the proximal end of the tubular irrigation member includes a locking mechanism for releasably connecting the irrigation member to the control handle.

8. The catheter according to claim 3 in which the first and the second elongate tubular sheath are axially displaceable in relation to one another.

9. The catheter according to claim 3 in which the first and the second elongate tubular sheath include two or more electrodes arranged at a predetermined distance from one another.

10. The catheter according to claim 3 wherein the displacement of the first or the second elongate tubular sheath is motorized.

11. The catheter according to claim 1 in which the distal end of the tubular irrigation member is heat set into a predetermined shape.

12. The catheter of claim 11 in which the predetermined shape is a loop shape.

13. The catheter of claim 1 in which the one or more perforations on the irrigation member are arranged in a predetermined pattern.

14. The catheter of claim 3 in which a steering element is inserted into the lumen of the first or the second elongate tubular sheath.

15. The catheter according to claim 1 in which conductors for the electrodes are contained within a wall of the tubular member.

16. The catheter according to claim 15 in which the conductors are wound helically between an inner and an outer layer of non-conductive material.

17. An ablation catheter which includes:
a tubular irrigation member of a non-conductive material having a proximal end and a distal end, the tubular member defining a fluid lumen and having one or more perforations at or proximal the distal end of the tubular member;
an elongate tubular sheath having a lumen extending from a proximal end of the tubular sheath to a distal end of the tubular sheath, the distal end of the elongate tubular sheath formed into a loop shape, the elongate tubular sheath having a plurality of ablation electrodes arranged at, or adjacent, the distal end, the elongate tubular member being removably received into the fluid lumen of the irrigation member and imposing the loop shape onto the irrigation member, and
a control handle, the proximal end of the elongate tubular sheath connected to a control handle.

18. The catheter according to claim 17 wherein the proximal end of the tubular irrigation member includes an irrigation connector for connecting the irrigation member on to the elongate tubular sheath.

19. The catheter according to claim 18 wherein the irrigation connector includes a sealing element and an inlet for receiving pressurized fluid into the fluid lumen of the irrigation member to be energized by the one or more ablation electrodes of the first or the second elongate tubular sheath before being expelled through the one or more perforations of the irrigation member.
